(19) United States
(12) Patent Application Publication
(10) Pub. No.: US 2021/0169678 A1
(43) Pub. Date: Jun. 10, 2021

(54) TUBE FOR ENSURING BREATHING DURING SLEEP

(71) Applicant: Rikio SASAKI, Akita (JP)

(72) Inventor: Rikio SASAKI, Akita (JP)

(21) Appl. No.: 16/768,690

(22) PCT Filed: Jun. 25, 2018
(86) PCT No.: PCT/JP2018/023927

§ 371 Date: Jun. 1, 2020

(30) Foreign Application Priority Data
Dec. 4, 2017 (JP) 2017-232333

(51) Int. Cl. A61F 5/56

(52) U.S. Cl. CPC A61F 5/56

(57) ABSTRACT

This tube for ensuring breathing during sleep can be inserted into a nasal cavity and the length of the tube can be adjusted depending on the manner of use during sleep. An internal thread of an adjustment cylinder is engaged with an external thread on a proximal end of a tube, and the adjustment cylinder is rotated to adjust the overall length of the tube with the adjustment cylinder attached thereto. A nostril cover is over a distal end of the tube from the side with a back-end open cylinder and slid to the adjustment cylinder so that the nostril cover and the adjustment cylinder are fitted together. The overall length of the tube for ensuring breathing during sleep with the adjustment cylinder attached thereto can be adjusted by rotating the adjustment cylinder depending on the manner of use during sleep.

### TECHNICAL FIELD

The present invention relates to a tube for ensuring breathing during sleep which is capable of performing an insertion into a nasal cavity without an abnormal feeling and allowing a fine adjustment of a tube length.

### BACKGROUND ART

Airways during sleep become narrowed or blocked due to not only obesity but also a skeleton of the face and advancing age. Snoring or apnea during sleep are caused by narrowing or closing of an upper airway. Therefore, inserting a soft tube into a nose to ensure the airway is open has been previously known.

The present inventor has developed a tube for ensuring breathing during sleep capable of performing an insertion into a nasal cavity without an abnormal feeling, and reliably attaching and detaching without dropping by a stopper that has no resistance in terms of an appearance (see Patent Document 1).

This known technique includes a tube for ensuring breathing during sleep which is a soft tube made of silicon rubber close to a human skin, and has at least a length from a nostril to a pharyngeal region.

A tip part of the tube for ensuring breathing during sleep is closed by a bell-shaped arc surface tapered toward the tip, and a small through hole is formed in a central portion of the arc surface to facilitate an air circulation, as well as an arc section curved in accordance with a shape from the nostril to the pharyngeal region is formed, and a plurality of vent holes linearly extending toward the tip part are formed along an inside ridgeline of the arc section.

The tube for ensuring breathing during sleep includes a truncated cone-shaped cylinder fixed to a base end part thereof and having a size to be fitted in the nostril, and a stopper formed on the cylinder and protruding in an arc shape along one quarter of a circumferential edge thereof. The stopper can be inserted into and supported to the nostril, thereby enabling reliably attaching and detaching the tube for ensuring breathing during sleep.

However, in the above tube for ensuring breathing during sleep, since the truncated cone-shaped cylinder having a size to be fitted in the nostril is fixed to the base end part thereof, and the stopper protruding in the arc shape along one quarter of the circumferential edge is formed on the cylinder, a specialized physician should accurately measure the length from the nostril to the pharyngeal region of a user and cut the tube to fix the cylinder, and it was not possible for the user to finely adjust the tube length depending on a using mode during sleep.

### PRIOR ART DOCUMENT

### Patent Document

[Patent Document 1] Japanese Patent Publication No. 6334801

### SUMMARY OF INVENTION

### Problems to be Solved by Invention

It is an object of the present invention to provide a tube for ensuring breathing during sleep which allows a user to perform an insertion into a nasal cavity without an abnormal feeling and to finely adjust a tube length depending on a using mode during sleep. MEANS FOR SOLVING PROBLEMS

The present invention provides a tube for ensuring breathing during sleep including: an adjustment cylinder and a nostril cover which are combined with each other, the tube for ensuring breathing during sleep is characterized in that: the tube for ensuring breathing during sleep is a tube for ensuring breathing during sleep which is a soft tube made of silicon rubber close to a human skin, and has at least a length from a nostril to a pharyngeal region, a tip part of the tube for ensuring breathing during sleep is closed by a bell-shaped arc surface tapered toward the tip, and a small through hole is formed in a central portion of the arc surface to facilitate an air circulation, as well as an arc section curved in accordance with a shape from the nostril to the pharyngeal region is formed, a vent hole extending toward the tip part are formed along an inside ridgeline of the arc section, a liquid passage hole is formed along an outer ridge of the arc section at a position shifted from the vent hole, and the tube for ensuring breathing during sleep has a male thread formed on a base end part thereof;

the adjustment cylinder provided with a female thread which is screwed to the male thread and configured to change an entire length of the tube for ensuring breathing during sleep by rotating the adjustment cylinder; and

a shell-shaped nostril cover having a size enough to be inserted into a nostril has a tip opening tube formed at a tip part thereof, into which the tube for ensuring breathing during sleep is inserted, and a distal opening tube integrally formed therewith at a distal part thereof to communicate with the tip opening tube, into which the adjustment cylinder is inserted.

### ADVANTAGEOUS EFFECTS

According to the present invention, there is provided a tube for ensuring breathing during sleep of including: an adjustment cylinder and a nostril cover which are combined with each other, the tube for ensuring breathing during sleep is characterized in that: the tube for ensuring breathing during sleep is a tube for ensuring breathing during sleep which is a soft tube made of silicon rubber close to a human skin, and has at least a length from a nostril to a pharyngeal region, a tip part of the tube for ensuring breathing during sleep is closed by a bell-shaped arc surface tapered toward the tip, and a small through hole is formed in a central portion of the arc surface to facilitate an air circulation, as well as an arc section curved in accordance with a shape from the nostril to the pharyngeal region is formed, a vent hole extending toward the tip part are formed along an inside ridgeline of the arc section, a liquid passage hole is formed along an outer ridge of the arc section at a position shifted from the vent hole, and the tube for ensuring breathing during sleep has a male thread formed on a base end part thereof;

the adjustment cylinder provided with a female thread which is screwed to the male thread and configured to change an entire length of the tube for ensuring breathing during sleep by rotating the adjustment cylinder; and

a shell-shaped nostril cover having a size enough to be inserted into a nostril has a tip opening tube formed at a tip part thereof, into which the tube for ensuring breathing during sleep is inserted, and a distal opening tube integrally formed therewith at a distal part thereof to communicate with the tip opening tube, into which the adjustment cylinder is inserted. Therefore, a user rotates the adjustment cylinder depending on a using mode (habit resulting from experience and a desire to insert the tip part of the tube for ensuring breathing during sleep to be a little deeper without an abnormal feeling), such that it is possible to finely adjust the entire length of the tube for ensuring breathing during sleep in which the adjustment cylinder is screwed.

### BRIEF DESCRIPTION OF DRAWINGS

FIG. 1 is an exploded explanatory view of a tube for ensuring breathing during sleep according to the present invention.

FIG. 2 is a front view of the tube for ensuring breathing during sleep of the present invention.

FIG. 3 is an enlarged longitudinal-sectional view illustrating a connection portion between the tube for ensuring breathing during sleep and an adjustment cylinder.

FIG. 4 is a perspective view of another embodiment of the tube for ensuring breathing during sleep according to the present invention.

FIG. 5 is a perspective view of another embodiment of the tube for ensuring breathing during sleep of the present invention.

### MODE FOR CARRYING OUT INVENTION

An embodiment of a tube for ensuring breathing during sleep of the present invention will be described below with reference to the accompanying drawings.

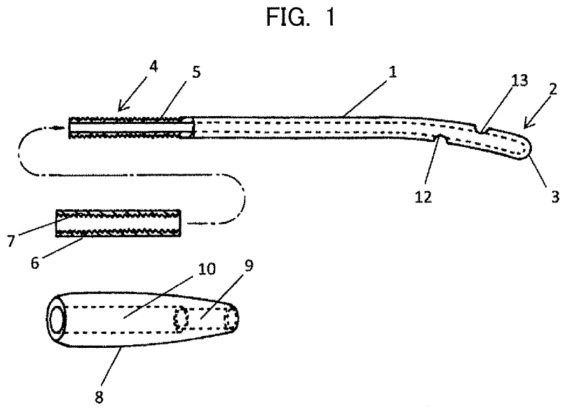

As shown in an exploded explanatory view of FIG. 1, a tube 1 for ensuring breathing during sleep is a soft transparent tube made of silicon rubber having softness close to that of a human skin, and has at least a length from a nostril to a pharyngeal region.

A tip part 2 of the tube 1 for ensuring breathing during sleep is closed by a bell-shaped arc surface 3 tapered toward the tip, and a small through hole (not illustrated) is formed in a central portion of the arc surface 3 to facilitate an air circulation. In addition, the tube 1 for ensuring breathing during sleep has a male thread 5 formed on a base end part 4 thereof, to which an adjustment cylinder 6 provided as a separate body for adjusting a length of the tube is screwed.

The adjustment cylinder 6 which is screwed to the male thread 5 has a female thread 7 formed therein, such that the entire length of the tube 1 for ensuring breathing during sleep may be changed by rotating the adjustment cylinder 6. In addition, although the base end part 4 has the male thread 5 and the adjustment cylinder 6 has the female thread 7, it may be configured so that the base end part 4 has the female thread 7 and the adjustment cylinder 6 has the male thread 5 in reverse.

Further, a shell-shaped nostril cover 8 is formed to have a size capable of being inserted into a nostril, which has a tip opening tube 9 formed at a tip part thereof, into which the tube 1 for ensuring breathing during sleep can be inserted, and a distal opening tube 10 integrally formed therewith at the distal part thereof to communicate with the tip opening tube 9, into which the adjustment cylinder 6 can be inserted.

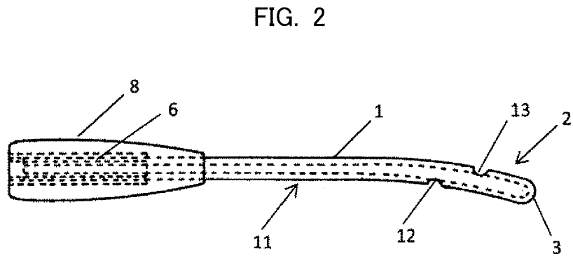

Then, as shown in a front view of FIG. 2, the tube 1 for ensuring breathing during sleep has a curved arc section 11 formed therein in accordance with a shape from the nostril to the pharyngeal region, wherein a vent hole 12 extending toward the tip part 2 is formed in the arc section 11 along an inner ridgeline thereof. Meanwhile, a liquid passage hole 13 is formed in the arc section 11 along an outer ridge thereof at a position deviated from the vent hole 12. Further, it is also possible to form a plurality of the vent holes 12 and a plurality of liquid passage holes 13, respectively.

Next, an operation of one embodiment of the tube for ensuring breathing during sleep according to the present invention will be described below with reference to the accompanying drawings.

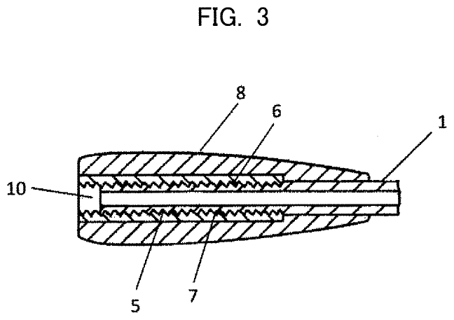

A user rotates the adjustment cylinder 6 by screwing the female thread 7 of the adjustment cylinder 6 to the male thread 5 provided on the base end part 4 of the tube 1 for ensuring breathing during sleep shown in FIG. 1, thereby the entire length of the tube 1 for ensuring breathing during sleep in which the adjustment cylinder 6 is screwed may be adjusted. When the length adjustment is completed, the user inserts the nostril cover 8 from the distal opening tube 10 side into the tip part 2 of the tube 1 for ensuring breathing during sleep, and as shown in an enlarged vertical sectional view of FIG. 3, slides the nostril cover 8 to the adjustment cylinder 6 so that they are completely fitted together.

In a state in which the tube 1 for ensuring breathing during sleep passes through the nasal cavity from the nostril and the nostril cover 8 of the tube 1 for ensuring breathing during sleep is placed in the nostril before going to bed, a user suffering from snoring or apnea pushes the above-described tube 1 for ensuring breathing during sleep up to the pharyngeal region. At this time, since the tube 1 for ensuring breathing during sleep has the bell-shaped arc surface 3 formed at the tip part 2, it is possible to insert the tube into the nasal cavity without damaging a mucous membrane without a feeling of hesitation. In particular, it is preferable for a beginner to apply petrolatum or a jelly-like lubricant to the tip part 2 of the tube 1 for ensuring breathing during sleep so as not to cause an abnormal feeling before insertion.

In this state, since the tip part 2 of the tube 1 for ensuring breathing during sleep is inserted into a deep side of the pharyngeal region to secure the airway, the air circulation is smoothly performed by the vent hole 12 on the inner ridgeline of the circular arc section 11 of the tube 1 for ensuring breathing during sleep and the through hole (not illustrated) in the central portion of the circular arc surface 3 of the tip part 2. In addition, a liquid such as runny nose is discharged from the liquid passage hole 13 on the outer ridge of the arc section 11 of the tube 1 for ensuring breathing during sleep.

At the time of getting up, when removing the tube 1 for ensuring breathing during sleep, the nostril is expanded with one hand, and the nostril cover 8 is pushed out from the outside of the nose with the other hand to project the tube from the nostril. By grasping and pulling out the nostril cover 8, the tube 1 for ensuring breathing during sleep can be removed.

In addition, the user rotates the adjustment cylinder 6 depending on the using mode (habit resulting from experience and a desire to insert the tip part 2 of the tube 1 for ensuring breathing during sleep to be a little deeper without an abnormal feeling), such that it is possible to finely adjust the entire length of the tube 1 for ensuring breathing during sleep in which the adjustment cylinder 6 is screwed.

Other embodiments of the tube for ensuring breathing during sleep according to the present invention will be described below with reference to the accompanying drawings.

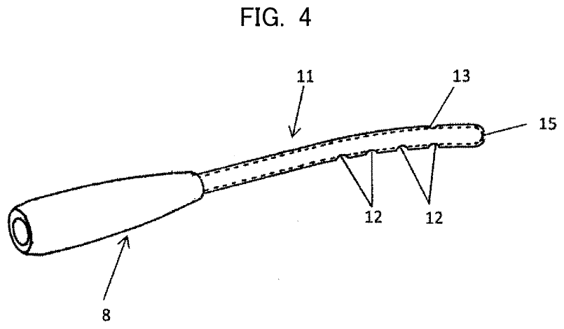

As shown in a perspective view of FIG. 4, a tube 1 for ensuring breathing during sleep is a soft transparent tube made of silicon rubber having softness close to that of a human skin, and has at least a length from a nostril to a pharyngeal region.

The tube 1 for ensuring breathing during sleep has a curved arc section 11 formed therein in accordance with a shape from the nostril to the pharyngeal region, wherein vent holes 12 extending toward the tip part 2 are formed in the arc section 11 along an inner ridgeline thereof. Meanwhile, a liquid passage hole 13 is formed in the arc section 11 along an outer ridge thereof at a position facing the vent hole 12.

In addition, a small through hole 15 is formed in a central portion of the arc surface 3 at the tip part 2 of the tube 1 for ensuring breathing during sleep. The through hole 15 is formed to facilitate the air circulation in the tube 1 for ensuring breathing during sleep.

Next, another embodiment of the tube for ensuring breathing during sleep of the present invention will be described below with reference to the accompanying drawings.

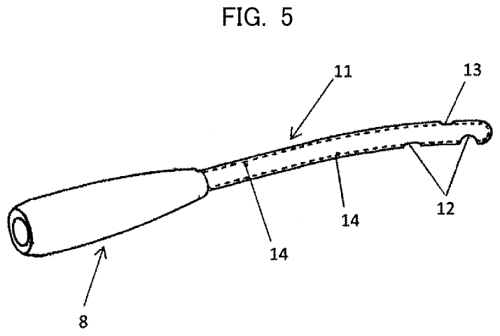

As shown in the perspective view of FIG. 5, a tube 1 for ensuring breathing during sleep is a soft transparent tube made of silicon rubber having softness close to that of a human skin, and has at least a length from a nostril to a pharyngeal region.

The tube 1 for ensuring breathing during sleep has a curved arc section 11 formed therein in accordance with a shape from the nostril to the pharyngeal region, wherein a plurality of small holes 14 extending spirally or randomly toward the tip part 2 side are formed in the arc section 11 along an entire length thereof.

In addition, the tube 1 for ensuring breathing during sleep has a plurality of vent holes 12 provided in the vicinity of the tip part 2 along the inner ridge of the arc section 11 thereof, and a liquid passage hole 13 provided on the outer ridgeline at a position deviated from the vent holes 12 of the tip part 2 thereof.

Further, the plurality of small holes 14 spirally or randomly extending along the entire arc section 11 toward the tip part 2 produces an effect of providing moisture in the tube 1 for ensuring breathing during sleep. In addition, a liquid such as runny nose is discharged from the liquid passage hole 13 on the outer ridgeline of the arc section of the tube 1 for ensuring breathing during sleep.

### DESCRIPTION OF REFERENCE NUMERALS

1 Tube for ensuring breathing during sleep

2 Tip part

3 Arc surface

4 Base end part

5 Male thread

6 Adjustment cylinder

7 Female thread

8 Nostril cover

9 Tip opening tube

10 Distal opening tube

11 Arc section

12 Vent hole

13 Liquid passage hole

14 Small hole

15 Through hole

1.-5. (canceled)

6. A tube for ensuring breathing during sleep comprising: an adjustment cylinder and a nostril cover which are combined with each other, the tube for ensuring breathing during sleep is characterized in that:
the tube for ensuring breathing during sleep is a tube for ensuring breathing during sleep which is a soft tube made of silicon rubber close to a human skin, and has at least a length from a nostril to a pharyngeal region, a tip part of the tube for ensuring breathing during sleep is closed by a bell-shaped arc surface tapered toward the tip, and a small through hole is formed in a central portion of the arc surface to facilitate an air circulation, as well as an arc section curved in accordance with a shape from the nostril to the pharyngeal region is formed, a vent hole extending toward the tip part is formed along an inside ridgeline of the arc section, and a liquid passage hole is formed along an outer ridge of the arc section at a position deviated from the vent hole;
the nostril cover has a tip opening tube formed at a tip part thereof, into which the tube for ensuring breathing during sleep is inserted, and a distal opening tube integrally formed therewith at a distal part thereof to communicate with the tip opening tube, into which the adjustment cylinder is inserted; and
an inner diameter of the distal opening tube is larger than an inner diameter of the tip opening tube, and the adjustment cylinder is engaged to a stepped portion between the distal opening tube and the tip opening tube.

7. The tube for ensuring breathing during sleep according to claim 6, wherein a plurality of vent holes extending toward the tip part side is formed in the arc section along an inner ridgeline thereof, and a liquid passage hole is formed in the arc section along an outer ridge thereof at a position facing the vent hole.

8. The tube for ensuring breathing during sleep according to claim 6, wherein a plurality of small holes extending spirally or randomly toward the tip part side are formed in the arc section along an entire length thereof.

9. The tube for ensuring breathing during sleep according to claim 8, wherein a plurality of vent holes are provided in the vicinity of the tip part along the inner ridge of the arc section of the tube for ensuring breathing during sleep, and a liquid passage hole is provided on the outer ridgeline at a position deviated from the vent holes of the tip part thereof.

10. The tube for ensuring breathing during sleep according to claim 6, wherein the entire tube for ensuring breathing during sleep is made of a transparent synthetic resin.

11. The tube for ensuring breathing during sleep according to claim 6, wherein the tube for ensuring breathing during sleep has a male thread formed on a base end part thereof, and the adjustment cylinder is provided with a female thread which is screwed to the male thread and is configured to change an entire length of the tube for ensuring breathing during sleep by rotating the adjustment cylinder

12. The tube for ensuring breathing during sleep according to claim 6, wherein the nostril cover is a shell shape nostril cover having a size enough to be inserted into a nostril.
